(19) United States
(12) Patent Application Publication
(10) Pub. No.: US 2024/0058202 A1
(43) Pub. Date: Feb. 22, 2024

(54) FOOT MASSAGE TOOL

(71) Applicant: Alleviate, Inc., Boston, MA (US)

(72) Inventors: Sean M. McDowell, Winchester, MA (US); Roderick McMullen, Boston, MA (US); Luke Ferdinands, Newton, MA (US)

(21) Appl. No.: 18/266,400

(22) PCT Filed: Dec. 10, 2021
(86) PCT No.: PCT/US21/62751

§ 371 Date: Jun. 9, 2023

Related U.S. Application Data
(60) Provisional application No. 63/123,697, filed on Dec. 10, 2020.

(51) Int. Cl. A61H 7/00; A61F 5/01

(52) U.S. Cl. CPC A61H 7/003; A61F 5/0127; A61H 2201/1253; A61H 2201/1695; A61H 2205/12

(57) ABSTRACT

A massage tool includes a rigid body having a first peak and a second peak, wherein the first peak and second peak are located approximately along a center line of the rigid body and there is a spacing between the first peak and the second peak. A height of the first peak may be different from a height of the second peak. A lower support structure may be disposed below the rigid body, shaped to provide a stable resting surface for the rigid body, and configured to accommodate one or more extendable support legs in a retracted position. A plurality of shallow depressions may form the surface of the rigid body. The one or more extendable support legs may be attached to a portion of the lower support structure, such as at a hinge point, and configured to retract under the rigid body.

### CROSS-REFERENCE TO RELATED APPLICATIONS

This application claims the benefit of U.S. Provisional Patent Application Ser. No. 63/123,697 (Attorney Docket No. ALLV-0002-P01), filed Dec. 10, 2020, and entitled “FOOT MASSAGE TOOL”.

Each of the foregoing applications is incorporated herein by reference in its entirety.

International (PCT) Patent Application No. PCT/US2021/033634 (Attorney Docket No. ALLV-0001-WO), filed on May 21, 2021, and titled “BRACE”, is incorporated herein by reference.

### BACKGROUND

### Field

The disclosure herein concerns, among other things, a foot massage tool.

### Description of the Related Art

When soft tissue (e.g., muscles, tendons, and ligaments) becomes inflamed, suffers trauma, becomes injured or is overtrained, scar tissue (e.g., fibrous adhesions) may form as part of the healing process which may lead to lost range of motion, weakness, pain, and decreased stability. While normal soft tissue (e.g., muscle tissue) typically displays linearly aligned muscle fibers, scarred or inflamed soft tissue appears non-linear or disoriented, and is weaker than normal, linear muscle fibers. Placing a load onto scarred, disoriented tissues can be painful. Myofascial intervention may be used in the therapy of scarred muscle tissue to disrupt the non-linear muscle fibers. Plantar fasciitis is an example of a soft tissue disorder of the foot caused by trauma, injury, overtraining, and the like that may benefit from massage.

Instrument assisted soft tissue mobilization (IASTM) and other myofascial intervention techniques (e.g., bodywork directed at the fascia, soft-tissue instrument-assisted mobilization, cross-friction massage, manual massage, deep friction massage, deep tissue massage) typically utilize tools (e.g., stainless steel tools) that an individual, such as a therapist, utilizes to grind scarred tissue. The tools are typically handheld, so having sufficient strength integrity to apply the appropriate amount of pressure in at least the hand wielding the tool is a barrier to use. Further, use of handheld tools to provide therapy for an extended period of time may cause hand injuries in the therapist. There remains a need for a therapeutic tool to aid in the therapy of scarred soft tissue (e.g., non-linear muscle fibers) that does not require use of hands.

### SUMMARY

In an aspect, a massage tool may include a rigid body having a first peak and a second peak, wherein the first peak and second peak are located approximately along a center line of the rigid body, the rigid body including a spacing between the first peak and the second peak. The height of the first peak may be different from a height of the second peak. A lower support structure may be disposed below the rigid body, shaped to provide a stable resting surface for the rigid body, and configured to accommodate one or more extendable support legs in a retracted position. A plurality of shallow depressions may at least partially cover the surface of the rigid body. The one or more extendable support legs may be attached to a portion of the lower support structure and configured to retract under the rigid body. A surface extending in the spacing between the first peak and the second peak forms an arc that accommodates a portion of a foot. A surface of the rigid body tapers bilaterally towards a center of the rigid body to form the first peak and the second peak. The one or more extendable support legs attach at a hinge point on the lower support structure. the one or more extendable support legs rotate on a hinge to extend away from the rigid body. The one or more extendable support legs include at least two sections connected at a hinge joint. The rigid body is made of at least one of wood, plastic, silicone, or metal.

A method of breaking up scar tissue from underlying soft tissue of a user by use of a massage tool including the steps of stabilizing a massage tool on a floor by extending one or more extendable support legs from below the massage tool, lubricating a surface of at least one of a skin of an affected foot and the massage tool, and grinding the affected foot along a surface of the massage tool in such a manner to loosen the scar tissue from the soft tissue.

An advantage of the disclosure herein is that operation of the floor-placed massage tool using body weight allows for greater pressure, or less pressure, to be applied to break up scar tissue with specificity of location, which increases efficiency of therapy and decreases discomfort to surrounding non-affected tissue. The foot may be rubbed or manipulated along the edge of a peak of the massage tool to break up scar tissue thereby obviating the need for a hand-wielded instrument. The massage tool may be used on any part of the body on which scar tissue has been built up, such as a result of the healing process. The particular soft tissue area illustrated herein, the plantar fascia, is merely illustrative of one possible application of the disclosed massage tool.

For example, in an embodiment, a massage tool is provided. The massage tool includes a rigid body and a support structure. The rigid body has a surface that includes a first peak having a first height and a second peak having a second height. The first peak and the second peak are located approximately along a center line of the rigid body, and the rigid body includes a spacing between the first peak and the second peak. The support structure disposed below the rigid body. The surface includes a plurality of at least one of depressions or projections.

In another embodiment, a method of loosening a scar tissue adhesion from underlying soft tissue of a user by use of a massage tool is provided. The method includes stabilizing the massage tool on a floor; and moving an affected foot of the user along a surface of the massage tool in such a manner so as to break up the scar tissue adhesion. The massage tool includes a rigid body having a first peak having a first height, and a second peak having a second height. The first peak and the second peak are located approximately along a center line of the rigid body, and the rigid body includes a spacing between the first peak and the second peak. Moving the affected foot along the surface of the massage tool includes moving the affected foot against at least one of the first peak or the second peak.

In yet another embodiment, a method of treating at least one of plantar fasciitis or another tendinopathy of the foot of a human patient is provided. The method includes placing the patient's affected foot on a massage tool having a surface with a plurality of at least one of projections or depressions; and applying a body weight of the patient to the patient's affected foot contemporaneously with moving the patient's affected foot over the plurality of at least one of projections or depressions so as to break scar tissue associated with the at least one of plantar fasciitis or other tendinopathy.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIGS. 1A and 1B depict perspective views of the massage tool, in accordance with an embodiment of the current disclosure;

FIG. 1C depicts a perspective view of the massage tool with extended support legs, in accordance with an embodiment of the current disclosure;

FIG. 1D depicts another perspective view of the massage tool, in accordance with an embodiment of the current disclosure;

FIG. 2A depicts a side view of the massage tool minus the extendable support legs, in accordance with an embodiment of the current disclosure;

FIG. 2B depicts an underside view of the massage tool minus the extendable support legs, in accordance with an embodiment of the current disclosure;

FIGS. 3A, 3B, and 3C depict the underside of the massage tool, in accordance with an embodiment of the current disclosure;

FIG. 4 depicts the massage tool in use, in accordance with an embodiment of the current disclosure;

FIG. 5A depicts two halves of an additional embodiment of the massage tool, in accordance with an embodiment of the current disclosure;

FIG. 5B depicts an additional embodiment of the massage tool in use, in accordance with an embodiment of the current disclosure;

FIG. 6A is a side view of an ankle brace that replicates a custom taping system, in accordance with an embodiment of the current disclosure;

FIG. 6B is a front view of an ankle brace that replicates a custom taping system, in accordance with an embodiment of the current disclosure;

FIG. 6C is a bottom view of an ankle brace that replicates a custom taping system, in accordance with an embodiment of the current disclosure;

FIG. 6D is a perspective view of an ankle brace that replicates a custom taping system, in accordance with an embodiment of the current disclosure;

FIG. 7 depicts a method of loosening a scar tissue adhesion from underlying soft tissue of a user by use of a massage tool, in accordance with an embodiment of the current disclosure; and

FIG. 8 depicts a method of treating at least one of plantar fasciitis or another tendinopathy of the foot of a human patient, in accordance with an embodiment of the current disclosure.

### DETAILED DESCRIPTION

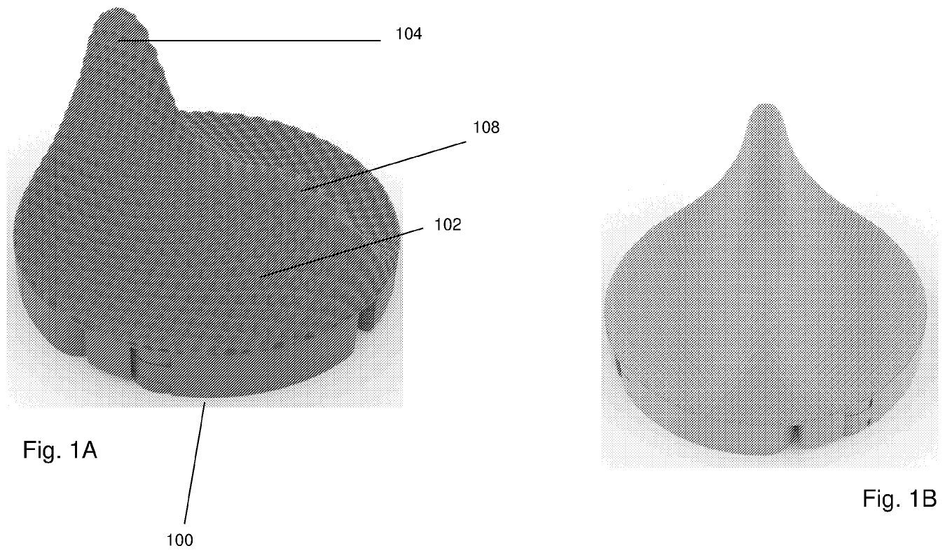

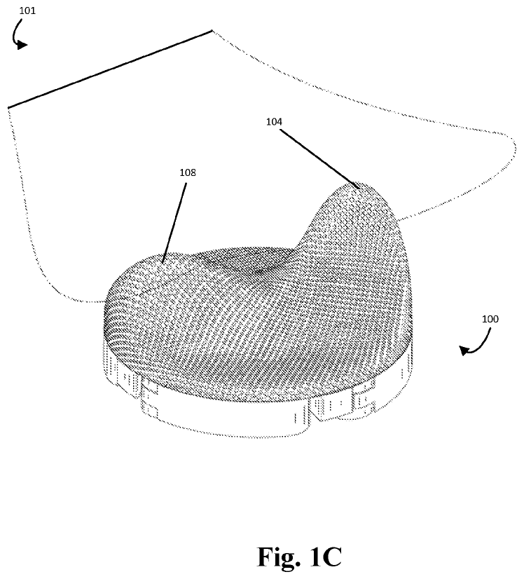

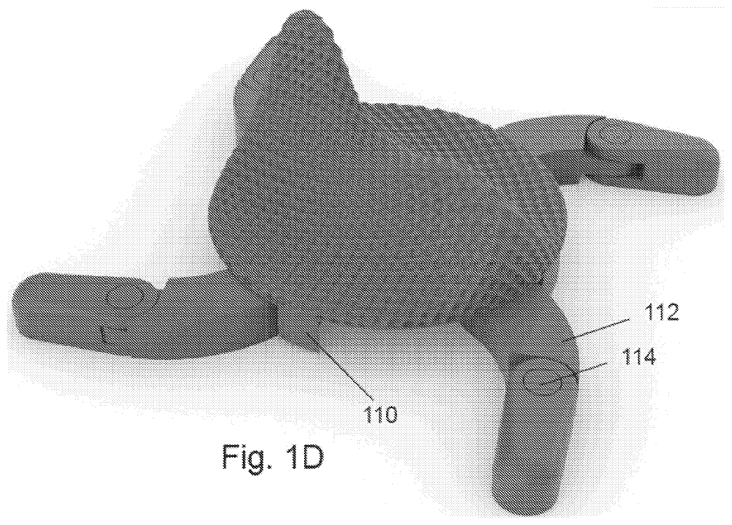

Referring to FIGS. 1A, 1B, and 1C, a massage tool 100 for a foot 101 (FIG. 1C) may include a rigid body 102 having a first peak 104 and a second peak 108, wherein the first peak 104 and second peak 108 are located in a row approximately along a center line of the rigid body 102. The surface of the rigid body may taper bilaterally towards a center of the rigid body to form the peaks. The peaks themselves may taper to an edge, such as a rounded edge or a blade edge. For example, a rounded edge of either of the peaks may have a small radius of curvature for a narrow peak or a large radius of curvature for a broader peak. In embodiments, the radius of curvature for each of the peaks may be different. Effectively, a width of the first peak 104 may be different from a width of the second peak 108. In embodiments, a height of the first peak 104 may be different from a height of the second peak 108. In some embodiments, there may only be one peak.

In an embodiment, the two mountains may be designed to anatomically fit the largest population of feet. Patients can get a deep penetration on the interior, exterior, posterior, anterior and plantar regions of the foot. The high peak can be for grinding scar tissue and helping to realign the fibers—which promotes healing, flexibility and freedom of movement. The valley allows patients to work the arch of the foot and the lower peak can be a broader, less aggressive penetration. The massage tool may be ambidextrous—it can be used on either the right or the left foot.

In embodiments, there may be a spacing between the first peak 104 and the second peak 108 such that, in one method of use, a user may place their foot in between the two peaks and rub it against one of the peaks. The surface extending in the spacing between the two peaks may form a curved portion, e.g., an arc, that accommodates a portion of a foot.

In embodiments, the geometry of the peaks, including their height, width, radius of curvature, inter-peak spacing, and orientation with respect to one another, may each be variable and may be variable with respect to each other. Certain geometries, such as a tall, narrow peak may be useful for more pinpoint pressure, while other geometries, such as a shorter and wider peak may be useful for individuals desiring less intense, pinpoint pressure. The massage tool may have any combination of peak geometry. For example, a massage tool may have two peaks of different height but the same radius of curvature. In another example, the peaks may be of different height as well as different radius of curvature. In an example, the peaks may be narrowly spaced while in another example, the peaks are spaced far apart. In an example, the peaks may be oriented in the same direction and may appear as if in a row. In another example, the peaks may be placed at an angle with respect to one another. It should be understood that the massage tool can have any combination of peak geometry, including peak spacing and orientation.

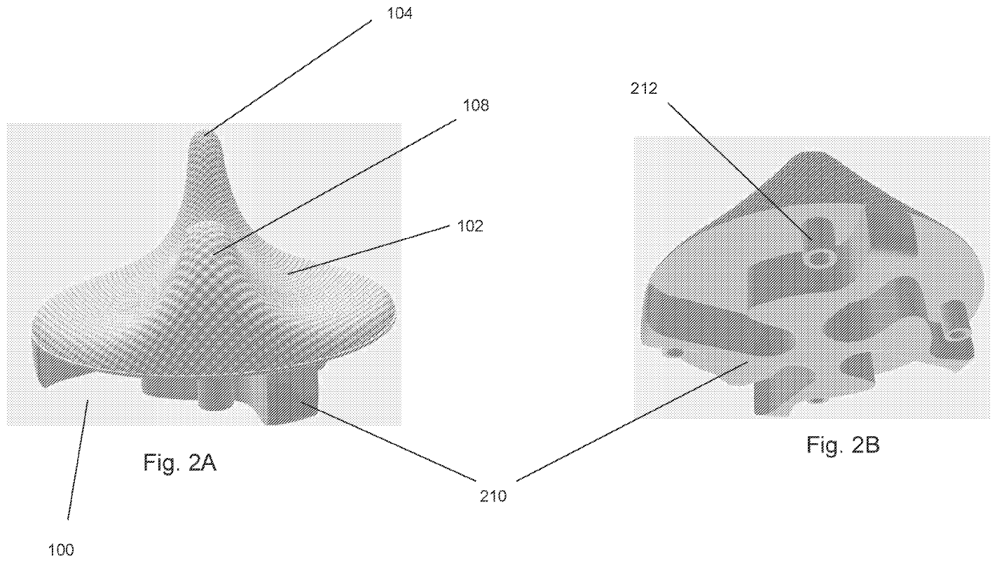

Referring briefly to FIG. 1D, a lower support structure 110 may be disposed below the rigid body 102 and may provide a stable resting surface for the massage tool. Referring now to FIGS. 2A and 2B, the lower support structure 210 may not cover the entirety of the lower portion of the rigid body 102, instead being configured to accommodate one or more extendable support legs 112 in a retracted position. FIGS. 2A and 2B eliminate the extendable legs for ease of viewing the lower support structure 210. The extendable support legs 112 may be attached to a portion of the lower support structure, such as at a hinge point 212. The extendable support legs 112 may rotate to extend away from the rigid body. In an embodiment, the one or more extendable support legs 112 may include at least two sections connected at a hinge joint 114. Being able to retract the support legs keeps the massage tool compact and portable. The massage tool may be used with the extendable support legs in the retracted or in the extended positions.

The retractable legs can expand to provide support when using the tool. Expanding the legs stabilizes the form when the foot is pressed down and keeps it from moving back and forth as the foot is grinding scar tissue. The retractable legs can retract into a compact form so the brace can be packed and stored easily, such as to fit into a gym bag and for shipping. The geometry of fitting in a circle and expanding solves the problem of portability, compactness, and additional support.

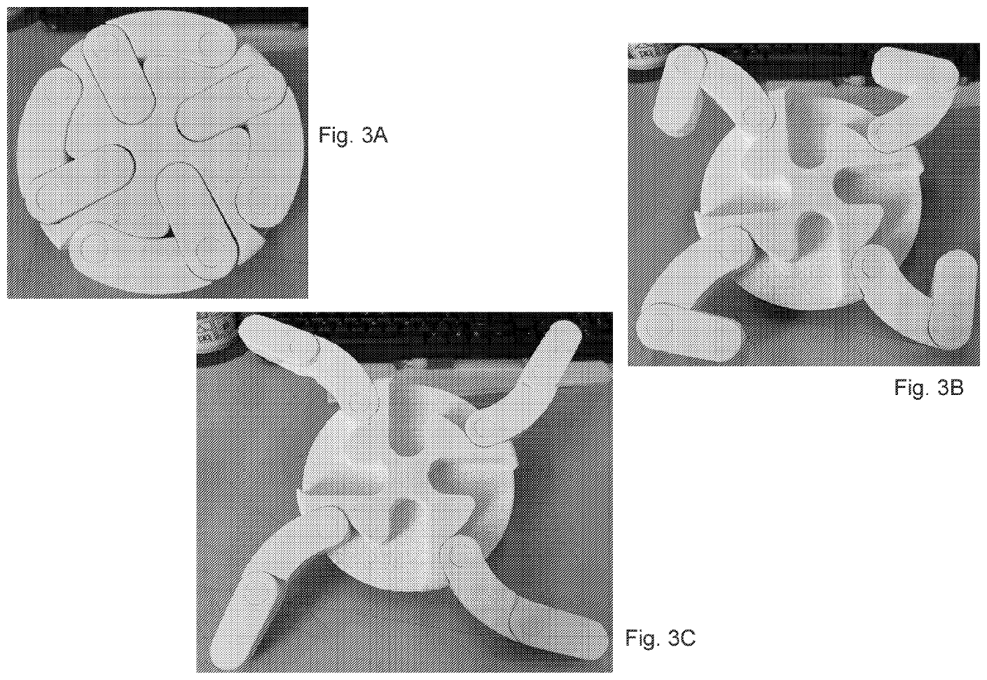

Referring now to FIGS. 3A, 3B, and 3C, the underside of the massage tool is depicted with the extendable support legs in place. In FIG. 3A, the extendable support legs are shown in their retracted state. In FIG. 3B, the extendable legs are partially extended and in FIG. 3C, the legs are fully extended.

In an embodiment, a surface of the rigid body 102 may include a plurality of shallow depressions or projections. The plurality of shallow depressions may resemble golf ball dimples, as shown in FIGS. 1A, 1B, 1C, 1D, and 2A. The dimpled surface may reduce surface friction so the skin can glide over the surface, such as by reducing the amount of surface area contacting the skin. Further, lubricant can be placed on the surface where it dwells in the shallow depressions and can be absorbed by the skin from the shallow depressions during use of the massage tool to enable smooth movement between skin and the massage tool, which may be important for users with numbness in their extremities or low skin integrity. In embodiments, only portions of the surface may have dimples. In embodiments, dimples may be spaced tightly or loosely. In embodiments, the dimples may be sized, in depth and circumference, to enable smooth gliding with the foot. For example, dimples may be 1-3 mm deep. In an embodiment, the dimples may be 1.5 mm deep. The diameter of the dimples may vary across the body of the tool. In an embodiment, the dimples may be 3-5 mm in diameter. In embodiments, dimples may be shallower at the top of the tools and deeper in the valley of the tool. Spacing of dimples may be uniform in some embodiments while variable in other embodiments. In some embodiments, the surface of the rigid body 102 includes a plurality of projections, such as rounded bumps, acupressure bodies, or the like.

In an embodiment, the rigid body 102 may be made of at least one of wood, plastic, silicone, or metal. In some embodiments, the entirety of the rigid body may be made from one material. In other embodiments, the rigid body may be made from more than one material. For example, one peak of the massage tool may be made from a plastic material while another peak may be made from a metal material. In another example, the tops of the peaks may be made from one material while the remainder of the rigid body comprises a different material. In embodiments, the rigid body, or the peaks of the rigid body, may be made from materials of different durometers. In embodiments, the rigid body, the peaks of the rigid body, the lower support structure, and the retractable legs may all be made from the same material or from materials having the same durometer. In embodiments, the rigid body, the peaks of the rigid body, the lower support structure, and the retractable legs may be made from different materials or from materials having different durometers.

In an embodiment, the area of the foot to be treated may be the plantar fascia where it originates in the heel and further along the foot where it turns into a muscular tendonous junction, as well as the inside of the arch. Use of the massage tool may be done in a standing or a sitting position, however, in the standing position, there may be an increased load on the plantar fascia that results in a flattening, wherein treatment of the flattened plantar fascia may be too irritating for some people. The massage tool may be placed on the floor for use. The extendable support legs may be in the retracted position beneath the rigid body, or may be extended to provide additional stability. Lotion or lubricant may be applied to the surface of the massage tool as well as on the underside of the foot to be treated. For example, an arnica lotion, which may also be therapeutic itself with respect to inflammation, may be applied to the tool and/or underside of foot.

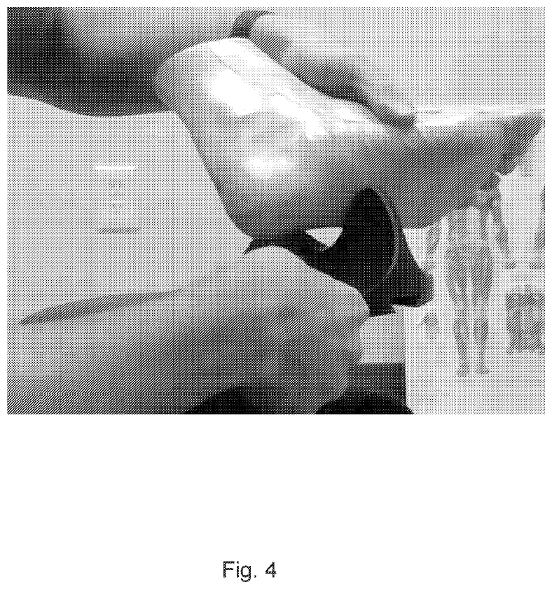

The user, in a seated or standing position, may grind or glide the treatment area of the foot, typically the heel and arch of the foot, on an edge of a peak of the massage tool, as depicted in FIG. 4. Either of the peaks of the massage tool can be used. A peak of a lower height or a more cambered peak (e.g., larger radius of curvature) may be more useful when beginning therapy or when the tissues are highly irritated.

In an embodiment, the massage tool may be used in one or more sessions, such as once or twice a day. In an embodiment, the massage tool may be used for similar or different durations during each use, such as between 3 and 5 minutes, with the goal of breaking down some of the scarring or non-linear tissue at the base of the foot, e.g., plantar fascia. The user's own body weight is used to break down scar tissue, applying more or less pressure as desired. The user may be able to locate scar tissue to treat by feeling the presence of a hardening of the underlying tissue and/or pain during use of the massage tool.

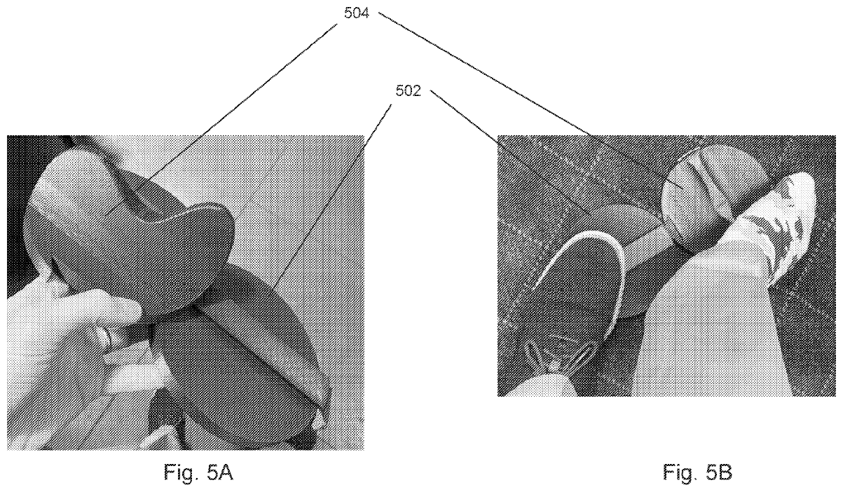

Breaking up scar tissue is accomplished by movement of the foot on the massage tool, such as depicted in FIG. 4 and FIG. 5B. As scar tissue begins to break up, irritation and swelling of the scar tissue may occur. The user may change the orientation of their foot with respect to the peaks during use such as for example to achieve a different result. In one example, using the massage tool in one orientation may break up the scar tissue, then altering the orientation of the foot during continued or subsequent use may help pull the scar tissue away from the underlying non-affected tissues.

FIGS. 5A and 5B depict another embodiment of the massage tool. FIG. 5A depicts the two pieces of the embodiment, an accessory support structure 502 and the rigid body 504, in an unassembled state. FIG. 5B shows the accessory support structure 502 and the rigid body 504 assembled. In the two-piece embodiment, the two pieces may come apart for transport or cleaning, and snap together for use. A cutout on the base of the rigid body 504 is interlocked or joined with a corresponding portion of the rigid body 504. In this embodiment, the user stands on or otherwise places a weight on the accessory support structure of the massage tool to stabilize it, while using the rigid body with the affected foot, as shown in FIG. 5B and as previously disclosed herein.

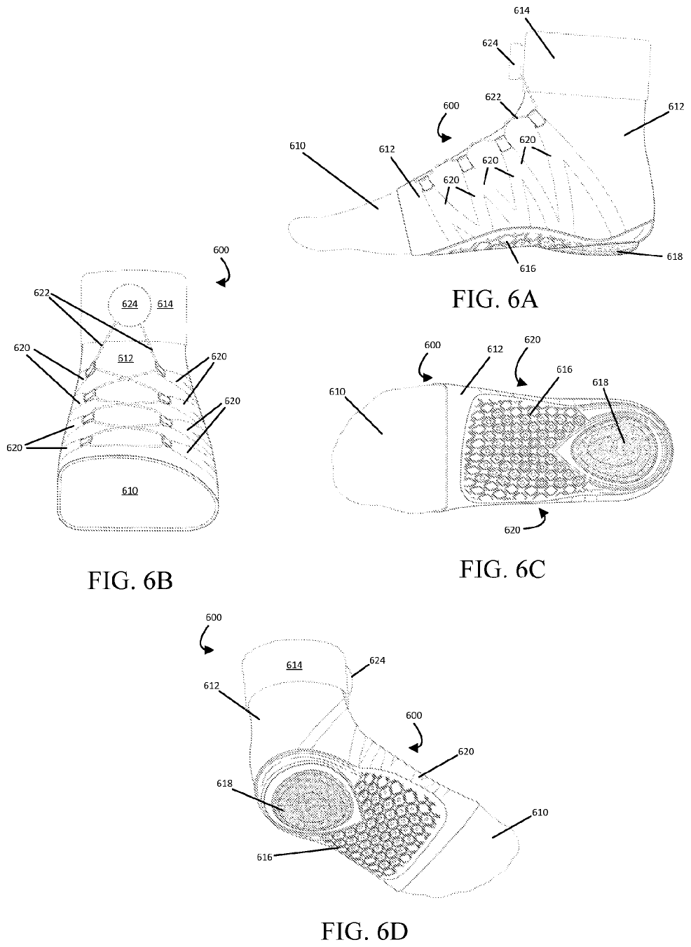

Turning to FIGS. 6A, 6B, 6C, and 6D, in an embodiment, the massage tool may be used as part of a complete therapeutic system. For example, an ankle brace 600 (depicted with a foot 610) that replicates a custom taping system and is effective at reducing foot and ankle dysfunction while allowing for foot and ankle mobility and stability may be used throughout the day to help unload the plantar fascia and enable user mobility. Then, the user may also use the massage tool in one or more sessions a day, each time for between 3 and 5 minutes. The goal is to unload the foot/plantar fascia with the brace in conjunction with using the massage tool to break up the scar tissue and/or pull scar tissue away from unaffected soft tissue. Finally, with the scar tissue broken up and loosened, stretches and exercises may be used to lengthen the muscles, tendons, and ligaments since the scar tissue has less of a hold on these underlying areas, optionally followed by icing or other inflammation-reducing measures. Further details of ankle braces in accordance with embodiments of the current disclosure may be found in International (PCT) Patent Application No. PCT/US2021/033634 (Attorney Docket No. ALLV-0001-WO), filed on May 21, 2021, and titled “BRACE”.

In an embodiment, a method of treating plantar fasciitis or another tendinopathy of the foot of a human patient may include placing the patient's affected foot on a massage tool having a surface with a plurality of projections or depressions; and breaking scar tissue associated with the plantar fasciitis or other tendinopathy by applying a body weight of the patient to the patient's affected foot contemporaneously with moving the patient's affected foot over the plurality of projections or depressions. The method may also include unloading a plantar fascia by wearing an ankle brace throughout a portion of a day. The ankle brace may include a knit portion 612 contoured to correspond to an ankle and foot structure, e.g., foot 610, and including an ankle strap 614 to secure the ankle brace 600 to a user's ankle; a rigid shank 616 adhered to the knit portion positioned on a bottom of a foot, wherein the rigid shank is configured to cover at least an arch portion of the foot; a pad 618 positioned below a heel of the foot and adhered to at least one of the knit portion 612 or the rigid shank 616; a webbing support system 620 wherein portions of the webbing support system 620 extend from a lateral extensor digitorum longus tendon around a peroneus longus retinaculum tendon, under a bottom of a user's foot to support a plantar tendon and up an arch and medial side of the user's foot to an extensor hallucis longus tendon; a lacing system 622 threaded through a portion of the webbing support system 620 at the top medial portion of the foot; and a tightening system 624 that applies a pulling force on the lacing system 622 to cinch the webbing support system 620.

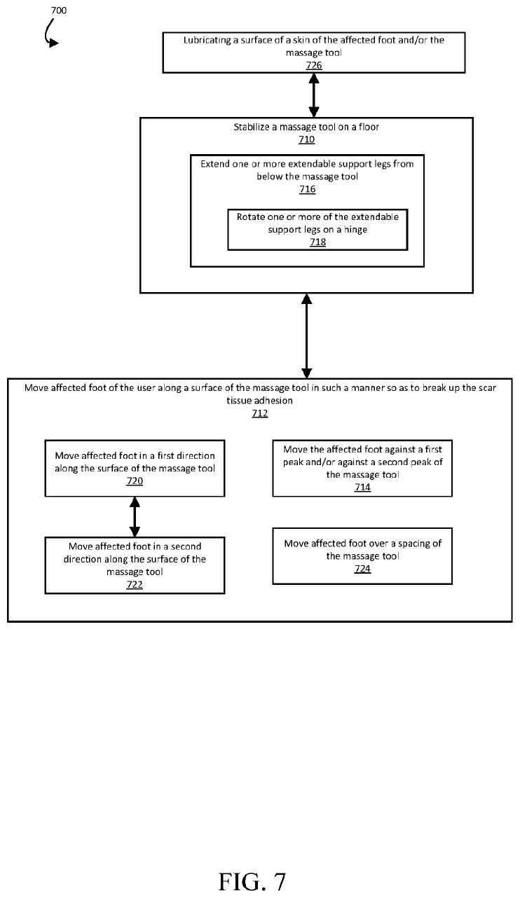

FIG. 7 depicts a method 700 of loosening a scar tissue adhesion from underlying soft tissue of a user, e.g., a patient, by use of a massage tool, in accordance with an embodiment of the current disclosure. As shown in FIG. 7, the method 700 may include stabilizing 710 a massage tool on a floor and moving 712 the affected foot of the user along a surface of the massage tool in such a manner so as to break up the scar tissue adhesion. Moving 712 the affected foot along the surface of the massage tool may include moving 714 the affected foot against a first peak and/or a second peak of the massage tool, for example, the peaks of the rigid body, as described herein. In embodiments, stabilizing 710 the massage tool on the floor may include extending 716 one or more extendable legs from below the massage tool, as also described herein. Extending 716 the one or more extendable support legs from below the massage tool may include rotating 718 the one or more extendable support legs on a hinge. In embodiments, moving 712 the affected foot along the surface of the massage tool may include moving 720 the affected foot in a first direction along the surface and subsequently moving 722 the affected foot in a second direction, which may be distinct from the first direction, along the surface. In embodiments, moving 712 the affected foot along the surface of the massage tool may include moving 724 the affected foot over a spacing that extends in the spacing between a first peak and a second peak, of the massage tool, so as to form a curved portion, e.g., an arch, that accommodates a portion of the affected foot, as also described herein. The method 700 may further include lubricating 726 a surface of at least one of a skin of an affected foot and the massage tool.

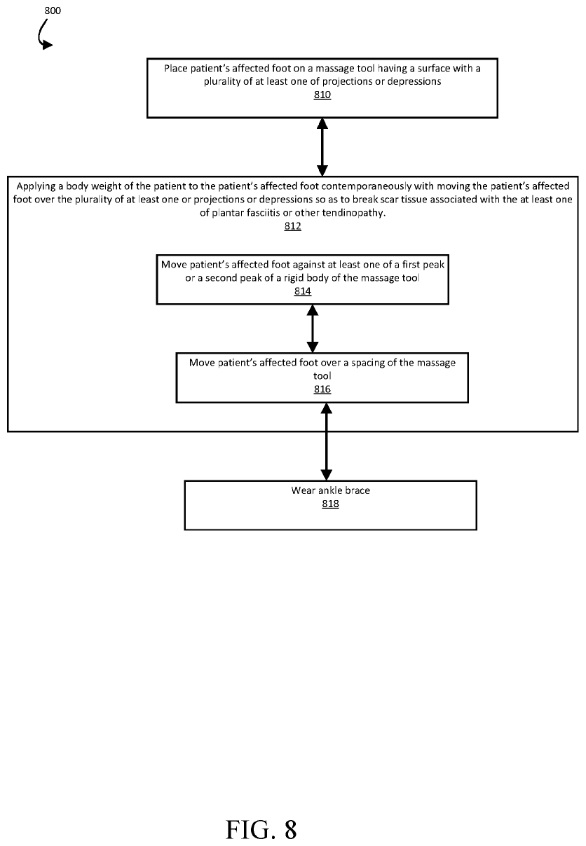

FIG. 8 depicts a method 800 of treating at least one of plantar fasciitis or another tendinopathy of the foot of a human patient. The method 800 may include placing 810 the patient's affected foot on a massage tool having a surface with a plurality of at least one of projections or depressions, and applying 812 a body weight of the patient to the patient's affected foot contemporaneously with moving the patient's affected foot over the plurality of at least one of projections or depressions so as to break scar tissue associated with the at least one of plantar fasciitis or other tendinopathy. In embodiments, the massage tool may include a rigid body having a first peak and a second peak, wherein the first peak and the second peak are located approximately along a center line of the rigid body, the rigid body including a spacing between the first peak and the second peak, wherein a height of the first peak may be distinct from a height of the second peak. In such embodiments, applying 812 a body weight of the patient to the patient's affected foot may include moving 814 the patient's affected foot against the first peak and/or the second peak. In embodiments, the surface may extend in the spacing between the first peak and the second peak and form a curved portion, e.g., an arc, that accommodates a portion of a foot. In such embodiments, applying 812 a body weight of the patient to the patient's affected foot may include moving 816 the affected foot over the spacing. In embodiments, the method 800 may include wearing 818 an ankle brace, e.g., 600 in FIGS. 6A-6D, throughout a portion of a day, which may unload a plantar fascia of the affected foot.

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entireties to the full extent permitted by law.

Without limitation to any other aspect of the present disclosure, aspects of the disclosure herein may provide for improved massage tools and methods of using the same.

For example, in an embodiment, a massage tool is provided. The massage tool includes a rigid body and a support structure. The rigid body has a surface that includes a first peak having a first height and a second peak having a second height. The first peak and the second peak are located approximately along a center line of the rigid body, and the rigid body includes a spacing between the first peak and the second peak. The support structure disposed below the rigid body. The surface includes a plurality of at least one of depressions or projections. In certain aspects, the first height is distinct from the second height. In certain aspects, the support structure is shaped to provide a resting surface for the rigid body. In certain aspects, the resting surface is structured to stabilize the rigid body. In certain aspects, a surface extending in the spacing between the first peak and the second peak forms a curved portion that accommodates a portion of a foot. In certain aspects, a portion of the surface of the rigid body tapers bilaterally towards a center of the rigid body to form, in part, the first peak and the second peak. In certain aspects, the support structure is configured to accommodate one or more extendable support legs in a retracted position. In certain aspects, the one or more extendable support legs are configured to move to the retracted position under the rigid body. In certain aspects, the one or more extendable support legs rotate on a hinge to extend away from the rigid body. In certain aspects, the one or more extendable support legs attach at a hinge point of the support structure. In certain aspects, the one or more extendable support legs comprise at least two sections connected at a hinge joint. In certain aspects, the rigid body is made of at least one of wood, plastic, silicone, or metal. In certain aspects, an edge of at least one of the first peak or the second peak tapers to at least one of a rounded edge or a blade edge. In certain aspects. the first peak and the second peak have a same radius of curvature. In certain aspects, the first peak and the second peak have a different radius of curvature. In certain aspects, the first peak comprises a first width and the second peak comprises a second width. In certain aspects, the first width is distinct from the second width. In certain aspects, at least one of the first peak and the second peak are positioned at an angle relative to the center line of the rigid body. In certain aspects, the following have a same durometer: the rigid body; the support structure; and one or more extendable support legs of the support structure. In certain aspects, at least two of the following have distinct durometers: the rigid body, the support structure, or one or more extendable support legs of the support structure.

In another embodiment, a method of loosening a scar tissue adhesion from underlying soft tissue of a user by use of a massage tool is provided. The method includes stabilizing the massage tool on a floor; and moving an affected foot of the user along a surface of the massage tool in such a manner so as to break up the scar tissue adhesion. The massage tool includes a rigid body having a first peak having a first height, and a second peak having a second height. The first peak and the second peak are located approximately along a center line of the rigid body, and the rigid body includes a spacing between the first peak and the second peak. Moving the affected foot along the surface of the massage tool includes moving the affected foot against at least one of the first peak or the second peak. In certain aspects, the first height is distinct from the second height. In certain aspects, stabilizing the massage tool on the floor includes extending one or more extendable support legs from below the massage tool. In certain aspects, extending the one or more extendable support legs from below the massage tool includes rotating the one or more extendable support legs on a hinge. In certain aspects, moving the affected foot along the surface of the massage tool includes moving the affected foot in a first direction along the surface and subsequently moving the affected foot in a second direction along the surface. In certain aspects, the first peak and the second peak have a same radius of curvature. In certain aspects, a surface extends in the spacing between the first peak and the second peak and forms a curved portion that accommodates a portion of the affected foot. Moving the affected foot along the surface of the massage tool further includes moving the affected foot over the spacing.

In yet another embodiment, a method of treating at least one of plantar fasciitis or another tendinopathy of the foot of a human patient is provided. The method includes placing the patient's affected foot on a massage tool having a surface with a plurality of at least one of projections or depressions; and applying a body weight of the patient to the patient's affected foot contemporaneously with moving the patient's affected foot over the plurality of at least one of projections or depressions so as to break scar tissue associated with the at least one of plantar fasciitis or other tendinopathy. In certain aspects, the massage tool includes a rigid body having a surface. The rigid body includes a first peak having a first height and a second peak having a second height. The first peak and the second peak are located approximately along a center line of the rigid body, and the rigid body includes a spacing between the first peak and the second peak. Applying the body weight contemporaneously with moving the patient's affected foot over the plurality of at least one of projections or depressions includes moving the patient's affected foot against at least one of the first peak or the second peak. In certain aspects, the surface extends in the spacing between the first peak and the second peak and forms a curved portion that accommodates a portion of the patient's affected foot. Moving the patient's affected foot over the plurality of at least one of projections or depressions includes moving the patient's affected foot over the spacing. In certain aspects, the method further includes comprising wearing an ankle brace throughout a portion of a day. In certain aspects, the ankle brace includes a knit portion, a rigid shank, a pad, a webbing support system, a lacing system, and a tightening system. The knit portion is contoured to correspond to an ankle and foot structure and includes an ankle strap to secure the ankle brace to a user's ankle. The rigid shank is adhered to the knit portion and positioned on a bottom of a foot, wherein the rigid shank is configured to cover at least an arch portion of the foot. The pad is positioned below a heel of the foot and adhered to at least one of the knit portion or the rigid shank. Portions of the webbing support system extend from a lateral extensor digitorum longus tendon around a peroneus longus retinaculum tendon, under a bottom of a user's foot to support a plantar tendon and up an arch and medial side of the user's foot to an extensor hallucis longus tendon. The lacing system is threaded through a portion of the webbing support system at the top medial portion of the foot. The tightening system applies a pulling force on the lacing system to cinch the webbing support system.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

While the foregoing written description enables one skilled in the art to make and use what is considered presently to be the best mode thereof, those skilled in the art will understand and appreciate the existence of variations, combinations, and equivalents of the specific embodiment, method, and examples herein. The disclosure should therefore not be limited by the above-described embodiment, method, and examples, but by all embodiments and methods within the scope and spirit of the disclosure.

Any element in a claim that does not explicitly state “means for” performing a specified function, or “step for” performing a specified function, is not to be interpreted as a “means” or “step” clause as specified in 35 U.S.C. § 112(f). In particular, any use of “step of” in the claims is not intended to invoke the provision of 35 U.S.C. § 112(f).

Persons skilled in the art may appreciate that numerous design configurations may be possible to enjoy the functional benefits of the inventive systems. Thus, given the wide variety of configurations and arrangements of embodiments of the present invention, the scope of the invention is reflected by the breadth of the claims below rather than narrowed by the embodiments described above.

What is claimed is:

1. A massage tool, comprising:
a rigid body having a surface, the rigid body comprising a first peak having a first height and a second peak having a second height, wherein the first peak and the second peak are located approximately along a center line of the rigid body, the rigid body comprising a spacing between the first peak and the second peak; and
a support structure disposed below the rigid body;
wherein the surface comprises a plurality of at least one of depressions or projections.

2. The massage tool of claim 1, wherein the first height is distinct from the second height.

3. The massage tool of claim 1, wherein the support structure is shaped to provide a resting surface for the rigid body.

4. The massage tool of claim 3, wherein the resting surface is structured to stabilize the rigid body.

5. The massage tool of claim 1, wherein a surface extending in the spacing between the first peak and the second peak forms a curved portion that accommodates a portion of a foot.

6. The massage tool of claim 1, wherein a portion of the surface of the rigid body tapers bilaterally towards a center of the rigid body to form, in part, the first peak and the second peak.

7. The massage tool of claim 1, wherein the support structure is configured to accommodate one or more extendable support legs in a retracted position.

8. The massage tool of claim 7, wherein the one or more extendable support legs are configured to move to the retracted position under the rigid body.

9. The massage tool of claim 8, wherein the one or more extendable support legs rotate on a hinge to extend away from the rigid body.

10. The massage tool of claim 8, wherein the one or more extendable support legs attach at a hinge point of the support structure.

11. The massage tool of claim 7, wherein the one or more extendable support legs comprise at least two sections connected at a hinge joint.

12. The massage tool of claim 1, wherein the rigid body is made of at least one of wood, plastic, silicone, or metal.

13. The massage tool of claim 1, wherein an edge of at least one of the first peak or the second peak tapers to at least one of a rounded edge or a blade edge.

14. The massage tool of claim 1, wherein the first peak and the second peak have a same radius of curvature.

15. The massage tool of claim 1, wherein the first peak and the second peak have a different radius of curvature.

16. The massage tool of claim 1, wherein the first peak comprises a first width and the second peak comprises a second width.

17. The massage tool of claim 16, wherein the first width is distinct from the second width.

18. The massage tool of claim 1, wherein at least one of the first peak and the second peak are positioned at an angle relative to the center line of the rigid body.

19. The massage tool of claim 1, wherein the following have a same durometer:
the rigid body;
the support structure; and
one or more extendable support legs of the support structure.

20. The massage tool of claim 1, wherein at least two of the following have distinct durometers:
the rigid body,
the support structure, or
one or more extendable support legs of the support structure.

21. A method of loosening a scar tissue adhesion from underlying soft tissue of a user by use of a massage tool, the method comprising:
stabilizing the massage tool on a floor; and
moving an affected foot of the user along a surface of the massage tool in such a manner so as to break up the scar tissue adhesion;
wherein the massage tool comprises:
a rigid body comprising:
a first peak having a first height, and
a second peak having a second height;
wherein the first peak and the second peak are located approximately along a center line of the rigid body, the rigid body comprising a spacing between the first peak and the second peak; and
wherein moving the affected foot along the surface of the massage tool comprises:
moving the affected foot against at least one of the first peak or the second peak.

22. The method of claim 21, wherein the first height is distinct from the second height.

23. The method of claim 21, wherein stabilizing the massage tool on the floor comprises:
extending one or more extendable support legs from below the massage tool.

24. The method of claim 23, wherein extending the one or more extendable support legs from below the massage tool comprises:
rotating the one or more extendable support legs on a hinge.

25. The method of claim 21, wherein moving the affected foot along the surface of the massage tool comprises:
moving the affected foot in a first direction along the surface and subsequently moving the affected foot in a second direction along the surface.

26. The method of claim 21, wherein the first peak and the second peak have a same radius of curvature.

27. The method of claim 21, wherein a surface extends in the spacing between the first peak and the second peak and forms a curved portion that accommodates a portion of the affected foot;
wherein moving the affected foot along the surface of the massage tool further comprises:
moving the affected foot over the spacing.

28. A method of treating at least one of plantar fasciitis or another tendinopathy of the foot of a human patient, the method comprising:
placing the patient's affected foot on a massage tool having a surface with a plurality of at least one of projections or depressions; and
applying a body weight of the patient to the patient's affected foot contemporaneously with moving the patient's affected foot over the plurality of at least one of projections or depressions so as to break scar tissue associated with the at least one of plantar fasciitis or other tendinopathy.

29. The method of claim 28, wherein the massage tool comprises:
a rigid body having a surface, the rigid body comprising a first peak having a first height and a second peak having a second height, wherein the first peak and the second peak are located approximately along a center line of the rigid body, the rigid body comprising a spacing between the first peak and the second peak;
wherein applying the body weight contemporaneously with moving the patient's affected foot over the plurality of at least one of projections or depressions comprises:
moving the patient's affected foot against at least one of the first peak or the second peak.

30. The method of claim 29, wherein the surface extends in the spacing between the first peak and the second peak and forms a curved portion that accommodates a portion of the patient's affected foot;
wherein moving the patient's affected foot over the plurality of at least one of projections or depressions comprises:
moving the patient's affected foot over the spacing.

31. The method of claim 28, further comprising wearing an ankle brace throughout a portion of a day.

32. The method of claim 31, wherein the ankle brace comprises:
a knit portion contoured to correspond to an ankle and foot structure and comprising an ankle strap to secure the ankle brace to a user's ankle;
a rigid shank adhered to the knit portion positioned on a bottom of a foot, wherein the rigid shank is configured to cover at least an arch portion of the foot;
a pad positioned below a heel of the foot and adhered to at least one of the knit portion or the rigid shank;
a webbing support system wherein portions of the webbing support system extend from a lateral extensor digitorum longus tendon around a peroneus longus retinaculum tendon, under a bottom of a user's foot to support a plantar tendon and up an arch and medial side of the user's foot to an extensor hallucis longus tendon;
a lacing system threaded through a portion of the webbing support system at the top medial portion of the foot; and
a tightening system that applies a pulling force on the lacing system to cinch the webbing support system.
